(19) United States
(12) Patent Application Publication
(10) Pub. No.: US 2007/0135859 A1
(43) Pub. Date: Jun. 14, 2007

(54) Use of transcranial magnetic stimulation to improve memory and stress related syndromes in humans

(76) Inventors: Peter Eriksson, Gothenburg (SE); Thorleif Thorlin, Gothenburg (SE)

Correspondence Address: LYNN E BARBER, P O BOX 16528, FORT WORTH, TX, 76162, US

(21) Appl. No.: 11/633,245

(22) Filed: Dec. 4, 2006

Related U.S. Application Data
(60) Provisional application No. 60/749,009, filed on Dec. 9, 2005.

(51) Int. Cl. A61N 1/00

(52) U.S. Cl. 607/42

(57) ABSTRACT

Use of rTMS having a range of stimuli frequency of 1-100 Hz, with 100-3000 pulses, given at each stimuli-session, and a stimulus intensity of 1-300 Ampere per microseconds, to induce certain electric fields in cells or tissue by the use of pulsed magnetic fields in healthy persons to modulate the proliferation, differentiation and/or migration of neural stem cells or progenitor cells in the adult central nervous system (CNS) especially in the hippocampal formation of dentate gyrus to improve memory and aid in improvement of stress-related syndromes, such as burnout.

### CROSS-REFERENCE TO RELATED APPLICATIONS

This is application claims priority from U.S. provisional application Ser. No. 60/749,009 filed Dec. 9, 2005, which is incorporated herein by reference.

### BACKGROUND OF THE INVENTION

1. Field of the Invention

The present invention relates to the use of pulsed magnetic fields, such as repetitive Transcranial Magnetic Stimulation (rTMS) or a functionally equivalent analogue to induce electric fields in cells or tissue by the use of pulsed magnetic fields, preferably from a handheld magnetic stimulator that upon exposure to a person's brain will induce proliferation, differentiation and/or migration of an embryonic stem cell, adult stem cell, progenitor cell and/or a cell derived from a stem cell or progenitor cell, especially in the hippocampal formation of dentate gyrus. The specific magnetic stimulation of the invention is preferably intended for healthy humans to improve memory or aiding in stress-related syndromes such as burnout.

2. Description of the Related Art

In most brain regions, the generation of neurons (neurogenesis) is generally confined to a discrete developmental period. Exceptions have recently been described in several regions of the brain that have been shown to generate new neurons well into the postnatal and adult period after a damage or disease. The best characterized regions are the subgranular zone (SGZ) of the dentate gyrus and the subventrical zone (SVZ) of the mammalian adult brain. This phenomenon is attributed to the existence of neuronal stem/progenitor cells.

Neuronal progenitor cells are stem cells and reside in the SGZ where they can proliferate, migrate into the granule cell layer and differentiate into granule cells. The new-born neurons in the granule cell layer express markers of differentiated neurons and have morphological characteristics corresponding to differentiated granule cells. Furthermore, they may establish axonal processes into the mossy fiber pathway and form synaptic connections with their targets in hippocampal layer CA3 (Gage, F. H., Science 287:1433-1438 (2000)). The disclosure of this publication, and of all other publications and patents referred to herein is incorporated herein by reference.

It has previously been shown that the proliferation of progenitor cells in the SGZ can be influenced by the administration of n-methyl-d-aspartate (NMDA) receptor antagonists or by the removal of the adrenal glands (Cameron, H. A. and Gould, E., Neuroscience 61: 203-209 (1994); Cameron, H. A., Tanapat, P. and Gould, E., Neuroscience 82: 349-354 (1998)). What is more, it was shown that exposure to an enriched environment leads to an increased number of surviving, newly formed granule cells as well as to an increased total number of neurons in the dentate gyrus (Kempermann, G., Kuhn, H. G. and Gage, F. H., Nature 386: 493-495 (1997)).

A way to stimulate the process of increasing the number of neurons in the brain would be of great interest due to the potential of replacing neurons following disease or damage. This is also of interest as neuronal progenitor cells are also thought to be important for the ability to learn, and our ability to cope with stress, and it also has been shown that adult neurogenesis is correlated with improved memory (Shorts T J et al, Nature. Mar. 15, 2001;410 (6826):372-6). Because neuronal plasticity is reduced with increased age, and studies have demonstrated that proliferation of progenitor cells is also decreased with age (Kuhn, H., Dickinson-Anson, H., and Gage, F. H., Journal: J. Neurosci. 16: pp 2027-2033 (1996)), it is also of interest to stimulate this activity in people of increased age.

Various works suggest that the ability of endogenous stem cells to proliferate, migrate, differentiate and integrate after damage can be enhanced by various stimulation signals. For example, administration of growth factors and electrical stimulation have each been suggested to promote neurogenesis and conceivably direct the proliferation, migration, differentiation and integration of new cells in the central nervous system after a lesion, for example described in U.S. Pat. Application No. 20050032702.

Burnout is a physical, mental, and emotional response to constant levels of high stress. Burnout produces feelings of hopelessness, powerlessness, cynicism, resentment and failure—as well as stagnation and reduced productivity. These stress reactions can result in levels of unhappiness that eventually threaten a person's job, relationships and health. Burnout is associated with situations in which a person feels overworked, underappreciated, confused about expectations and priorities, concerned about job security, overcommitted with responsibilities, and resentful about duties that are not commensurate with pay.

Burnout can occur when a person feels unable to meet constant demands, and becomes increasingly overwhelmed and depleted of energy. Debilitating sadness, anger or indifference can set in. The person with burnout begins to lose the interest or motivation that led the person to take on a certain role in the first place. Burnout is generally recognized as a work-related stress-induced condition associated with memory problems, fatigue, a sense of inadequacy, and depressed moods. It is not considered to be a disease. Eriksson et al. (2004) (Journal: Acta Neurologica Scandinavica, Volume 110, Number 5, November 2004, pp. 275-280(6)) propose burnout to be an exponent of stress-mediated decrease in adult neurogenesis leading to a decreased ability to cope with stress through decreased hippocampal function possibly involving a disturbed hippocampal regulation of the hypothalamo-pituitary-adrenal (HPA) axis.

A recent theory on the pathophysiology of depression involves disturbances in the hippocampal neurogenesis as a causative factor in development of the depressive disease. This theory is in part based on the observation that i) depressed patients present smaller hippocampal volumes, ii) anti-depressive treatments increase neurogenesis in animal models, and iii) increased levels of cortisol, which is known to be important factor in stress-induced depression, also are strong inhibitors of the neurogenesis. (B. L. Jacobs, H. Praag, F. H. Gage, Journal: Mol. Psychiatry; 5: p 262 (2000).

In patients with Major Depressive Disorders, transcranial magnetic stimulation (TMS) applied to the appropriate regions and with appropriate stimulation parameters has shown antidepressant effects. TMS has now been approved as a treatment for depression in a wide variety of countries. The mechanisms involved are still elusive.

Since its introduction in 1985, TMS has also become firmly established as a useful diagnostic and investigative tool in clinical neurophysiology. TMS has also become increasingly popular as a probe in the exploration of normal human brain physiology and the correlates between brain activity and behaviour.

A growing number of studies suggest that TMS may have a place in the treatment of a range of neurologic diseases including depression, Parkinson's disease (Pascual-Leone A, et. al. Akinesia in Parkinson's disease, Neurology 1994; 44; 884-891; Mallory J, Stone T. Repetitive transcranial magnetic stimulation induces an improvement in parkinsonian symptoms. Medical Science Research 1998, 26; 521-523.; Málly J, et. al. Long-term follow up-study with rTMS in Parkinson's disease. Brain Res Bull 2004; 259-63.), writer's cramp (Siebner H R, Mentschel C, Auer C, Conrad B. rTMS has a beneficial effect on bradicinesia in Parkinson's disease. Neuroreport 1999b; 10; 589-94), epilepsy (Tergau F, Naumann U, Paulus W, Steinhoff B. Low-frequency repetitive transcranial magnetic stimulation improves intractable epilepsy. Lancet 1999; 353; 2209.) or stroke recovery (Mansur C G, Fregni F, Boggio P S, Riberto M, Gallucci-Neto J, Santos C M, Wagner T, Rigonatti S P, Marcolin M A, Pascual-Leone A. A sham-stimulation controlled trial of rTMS of the unaffected hemisphere in stroke patients Neurology 2005; 64; 1802-1804). However, such suggestions of therapeutic potential of TMS in neurologic disease are very preliminary and much experimental work is still needed to assess their practical significance.

The background to TMS logically follows the discovery of the unified and interchangeable nature of electric and magnetic forces, from which it became evident that electric currents generate magnetic fields while changing magnetic fields generate electric currents. By this time it had already been well established by Galvani's and Volta's classic experiments that electric currents were capable of stimulating neuronal tissues. The combination of these two concepts—the unity of electric and magnetic forces, and the responsiveness of neurons to electrical stimulation—has been used to study and manipulate the central nervous system (CNS) to treat various diseases from both neurologic and psychiatric perspectives.

TMS uses an externally generated changing magnetic field to induce electric current intra-cranially. This is in contrast to the application of an electric current that is generated externally and transmitted to the brain through the skull (for example in electroconvulsive therapy-ECT). When electricity is forced to pass through the skull, the current used must be relatively large as the skull is a powerful insulator with an electrical resistance 8 to 15 times greater than that of soft tissues. Furthermore, externally generated electric currents cannot be focally directed as the skull dissipates the electricity globally, leading to massive depolarization of cortical and subcortical structures. Such difficulties are minimized upon exposure of the skull to TMS, where the changing external magnetic field undergoes minimal attenuation in the skull tissues while inducing smaller, focally directed electric currents within the brain.

In short, TMS uses the principle of inductance to get electrical energy across the scalp and skull without the pain of direct percutaneous electrical stimulation. It involves placing a small coil of wire on the scalp and passing a powerful and rapidly changing current pulse through it. This produces a magnetic field, which passes relatively unimpeded through skin, scalp, and skull, and is tolerated well by most subjects.

Single-pulse TMS refers to single stimuli to a given brain region every 5 to 10 second. Repetitive stimulation, termed rTMS, can be slow or fast. Slow (or low frequency) rTMS refers to stimulation at a frequency of 1 Hz or less. Fast (or high frequency) rTMS refers to stimulation at rates above 1 Hz (Wassermann E M. Electroencephalogr Clin Neurophysiol 1998; 108; 1-16).

Electroconvulsive therapy (ECT) is a well-known treatment for depression, especially for the medication-resistant forms and those with psychotic symptoms. Further, major depression is often associated with elevated glucocorticoid levels. High levels of glucocorticoids reduce neurogenesis in the adult rat hippocampus. ECT has been postulated to enhance neurogenesis from a reduced level in disease. Hellsten et. al. (European Journal of Neuroscience Vol 16:2 P. 283, July 2002) conclude that ECT can increase hippocampal neurogenesis even in the presence of elevated levels of glucocorticoids. This further supports the hypothesis that induction of neurogenesis is an important event in the action of antidepressant treatment. However ECT needs anaesthesia and muscle relaxation and is, very importantly and to the contrary of the present invention, associated with memory impairment.

The electrical currents resulting from TMS can be applied focally, without inducing a generalized convulsion because electromagnetism allows a reliable bridge across the skull. Zyss T et al, (Biol Psych 1997; 42; 920-924) published preliminary data comparing behavioral and biochemical effects of electroconvulsive stimulation (ECT) and TMS in rats, suggesting that both might involve similar mechanisms of action in this respect.

Possible long-term deleterious effects of TMS on cognition have been a concern since the beginning of its application as both a research and a clinical tool. Healthy volunteers are frequently exposed to single pulse or repetitive TMS during experiments in neuroscience. Attention, memory, executive functions and motor processing have been examined in several studies to ensure that no deleterious effect on cognition can be attributed to TMS (Bridgers and Delaney, Neurology 1989; 39; 417-9), (Hufnagelet al., J Neurol 1993; 240; 373-6), (Jahanshahi et al., Electroenceph Clin Neurophysiol 1997; 105; 422-9. Moreover, improvements in cognitive and motor performance have been reported (Siebner et al., Neurology 1999; 52; 529-537), (Mottaghy, et al., Neurology 1999; 53; 1806-12.). Pascual-Leone et al (Electroencephalograpy and Clinical Neurophysiology (1993) 89:120-130) has reported a study in healthy volunteers where they conclude that rTMS, as applied in their hands, was not associated with significant changes in among other things, cognitive performance.

The well-known ECT side effects were compared to TMS in some studies (O'Connor et al., Cogn Behav Neurol 2003; 16; 118-27). Cognitive evaluations showed transient disruptive effects of ECT on various aspects of memory, and a permanent retrograde amnesia. TMS did not exert any deleterious effects on memory.

In summary, no cognitive effects, positive or negative, of rTMS has so far been reported in healthy or in depressed humans.

Regarding the background of the equipment used in the invention herein, Barker et al. (Lancet 1:1106-1107) first described in 1985 the use of a pulsed (i.e., changing) magnetic field focused over specific regions of the cerebral cortex to induce muscle action potentials. The use of pulsed magnetic fields to induce electrical activity in peripheral nerves had been described much earlier in the 1960's. The mathematical framework describing how pulsed magnetic fields may be used to generate electrical currents in the human brain was subsequently described by Barker in 1987 (Neurosurgery 20:100-109).

The TMS technique requires a hand-held coil, for example, a coil shaped as a circular disc, with an inner diameter of approximately 60 millimeters (mm) and an outer diameter of approximately 130 mm. The coil is held near the patient's head, and is connected to a power-source which generates an electric current that is switched on and off repeatedly producing a changing magnetic field in the vicinity of the coil. The frequency at which the current (and hence magnetic field) is pulsed varies from as low as 1-5 Hz to as high as 25-30 Hz and even up to 100 Hz. rTMS is believed to be unique in that rapid pulsation can induce electrical currents within neurons while they are in the refractory period, although how this relates to an altered clinical manifestation is unclear. Being a relatively new technique, optimization of parameters such as frequency of pulsing of the magnetic field, size of the coil utilized, strength of the magnetic field generated, and duration of induction of electrical current has yet to be established. Since the initiation of the use of rTMS, more practical work has been done to establish parameters for treatment of disease using rTMS, but no one has previously done work on healthy persons as in the invention, nor has anyone established optimum parameters for application on healthy persons, for example to improve memory or aid in improving stress-related syndromes, such as burnout.

Some examples of hand held magnetic stimulators are, Magstim® Rapid by The Magstim Company Ltd (Spring Gardens, Whitland, Carmarthenshire, Wales, U.K) a magnetic stimulator that combine stimulation frequencies from 1 Hz to 100 Hz with a touch screen interface, which controls every aspect of the stimulator's control and operation. 1 Hz to 100 Hz stimulation frequency is achievable (100% output up to 25 Hz, 50% output at 50 Hz, 30% output at 100 Hz). All controls are operated via a dedicated TFT/VGA touch-screen. Pulses available are: single pulse, repetitive and session modes. Integrated 2 channel EMG with acquisition software includes latency and amplitude cursors. According to the manufacturer the stimulators are designed for use in: depression, rehabilitation, epilepsy, movement disorders, and functional brain mapping.

Another example of a TMS device is the MagPro-series by Medtronic (Shoreview, Minn., USA) which is a high performance magnetic stimulator for use in both the neurology clinic and in medical research. MagPro X100 is able to stimulate with repetition rates up to 100 Hz, and precise studies of the field/nerve interface can be done using the ability to choose monophasic or biphasic waveform and coil-current direction. The MagPro-series is equipped with a built-in trigger source, offering a selection of train durations (“train” is a sequence of pulses and “train-duration” the length in time of a “train”) from 0.2 to 10 seconds with repetition rates ranging from 5 to 100 pulses per second. The built-in source enables fast and easy set-up and the use of MagPro as a stand-alone device. Manual and external trig in/out is provided as well. The easy-to-use MagTrig program enables user configured stimulation protocols. The MagTrig program can be installed on any PC. The MagPro has an LED display showing the realized current gradient providing a precise and reproducible value for the stimulation strength. The coil temperature is displayed on an easy to read 6 level bar graph, and as for all their stimulators, MagPro has a build-in thermo-sensor that prevents triggering if the coil temperature exceeds 40° C.

Functional magnetic coils are produced in a variety of shapes including circles, figure eight, squares, petals, and spirals. See, e.g. Caldwell, J., Optimizing Magnetic Stimulator Design, Magnetic Motor Stimulation: Basic Principles and Clinical Experience, 1991, 238-48 (ed. Levy, W. J., et al.); Zimmermann, K. P., and Simpson, R. K., Electroencephal. Clin. Neurophysiol., 101:145-52 (1996); and U.S. Pat. No. 6,066,084 (Edrich et al.). The coils may include features other than a coil of a transducing material. For example, U.S. Pat. No. 6,086,525 (Davey et al.) and WO 98/06342 (Epstein et al.) disclose magnetic stimulators made from coil windings around a core of ferromagnetic material, preferably vanadium permendur. However, such coils affect cortical regions of the brain. U.S. Pat. Application No. 20040078056 describes a magnetic coil capable of stimulating the deep regions of the brain.

Studies by Czeh et al. (Biol Psychiatry 2002; 52(11): 1057-65) using low doses of rTMS support the notion that attenuation of the hypothalamic-pituitary-adrenocortical system is an important mechanism underlying the clinically observed antidepressant effect of rTMS, whereas the experimental design did not reveal beneficial effects of rTMS on adult hippocampal neurogenesis, even though the researchers looked for these.

In 2004, Arias-Carrion et al (Journal of Neuroscience Research 78:16-28 (2004) reported the induction of neurogenesis in the sub-ventricular zone (SVZ) and the differentiation after damage such as a nigrostriatal pathway lesion along with transcranial magnetic field stimulation (TMFS) in rats. This technique uses magnetic fields at the mT (milli-tesla) level not at the Tesla level of the present invention. Unlike the invention herein, they do not disclose neurogenesis in hippocampal dentate gyrus, nor in healthy humans, nor the specific magnetic stimulation needed for improving memory or aiding in stress-related syndromes such as burnout.

U.S. Pat. Application No. 20050119712 reveals the method of combining several different approaches simultaneously or in sequence to promote neurogenesis such as electrical signals, chemical agents or cell enhancing agents. The disclosure describes devices and methods to treat disease trough promoting recovery of damaged CNS tissue. However neither this device nor any of the prior art mentions neurogenesis stimulated only by rTMS in healthy humans.

The presence of ongoing neurogenesis in the healthy adult mammalian brain makes it possible to stimulate endogenous progenitor cells to be better able to generate new neurons, specifically in the hippocampal area, not only to replace cells lost through brain injury or neurodegenerative disease but also to improve memory or aiding in stress related syndromes of healthy persons.

Several researchers have demonstrated improved cell proliferation and the generation of new neurons in various diseased brains but none describe or suggest the application of rTMS to healthy humans for improving memory or aiding in stress-related syndromes, such as burnout.

Other objects and advantages of the invention herein will be more fully apparent from the following disclosure.

### SUMMARY OF THE INVENTION

The present invention relates to the unexpected finding that rTMS having a range of stimuli frequency of 1-100 Hz (preferably 20-100 Hz), with 100-3000 pulses, preferably more than 200 pulses, and most preferably more than 1000 pulses given at each stimuli-session, and a stimulus intensity of 1-300 Ampere per microseconds preferably 10-100 Ampere per microseconds, induce certain electric fields in cells or tissue by the use of pulsed magnetic fields, and that this specific stimulation is optimal in healthy persons in modulating the proliferation, differentiation and/or migration of neural stem cells or progenitor cells in the adult central nervous system (CNS) especially in the hippocampal formation of dentate gyrus.

Thus, one object of this invention is to provide new and better means to improve memory and aid in stress-related syndromes, such as burnout.

Another object of the present invention relates to the use of rTMS at a specific range of stimuli or a functionally equivalent analogue to induce electric fields in cells or tissue for improving memory or treat stress-related syndromes, such as burnout, by the use of pulsed magnetic fields using a technical device mentioned herein that induces proliferation, differentiation and/or migration of an embryonic stem cell, adult stem cell, progenitor cell and/or a cell derived from a stem cell or progenitor cell

### BRIEF DESCRIPTION OF THE DRAWINGS

FIG. 1 illustrates the experimental paradigm used in the experiments set forth herein.

FIG. 2 consists of six diagrams. Diagram (A) shows the increase in number of new born neuronal progenitors (BrdU positive cells) in the hippocampus of brains treated with rTMS of various number of pulses as compared to control and sham treatments. The brains are analyzed one day after the last rTMS treatment and show rTMS induced proliferation in hippocampus (see text for details). Diagram (B) is rTMS treated brains analyzed four weeks after last rTMS treatment, showing that the increase in cell numbers during rTMS seen in (A) also remains four weeks after rTMS is finished. Diagram (C) shows the number of new born cells that become neurons (NeuN) and astrocytes (GFAp) after four weeks of maturation. The graph shows that 80 of the new born cells in the hippocampus becomes neurons after the rTMS treatment. The diagrams (A-C) shows that rTMS stimulation of the brain leads to neurogenesis, which is the generation of new neurons. Diagram (D) shows the increase in hippocampal MAPK in rTMS treated brains. Diagram (E) shows the stimulation of pCREB in hippocampus by rTMS treatment, and in diagram (F) the number of pCREB positive cells is correlated to the number of new born neuronal progenitors (BrdU), showing a significant correlation between these parameters.

### DETAILED DESCRIPTION OF THE INVENTION AND PREFERRED EMBODIMENTS THEREOF

As discussed in more detail above, the mammalian brain, including the human brain, retains its ability to generate neurons throughout life, but in only certain regions. New neurons and astroglial cells and oligodendrocytes are generated by cell genesis from stem or progenitor cells. During the research leading to the present invention, it was found that rTMS induces an increase in cell genesis from progenitors and/or stem cells in the adult brain. In brief, the invention herein utilizes rTMS to stimulate neurogenesis in a human brain, in particular, in the hippocampal formation of dentate gyrus, especially in healthy humans, for example, to improve memory, aid in stress-related burnout and the like, as described in more detail below.

The invention herein provides a mechanism to treat neural loss suffered after a CNS insult or in the progress of a neuronal disease or disorder, by increasing the number of stem cell or progenitor derived cells, including neurons, astroglial cells and/or oligodendrocytes, by administering an effective amount of rTMS or a functionally equivalent analogue to induce electric fields in cells or tissue by the use of pulsed magnetic fields, to the patient with the intention to induce proliferation and/or differentiation of stem cells or progenitor cells with a concomitant increase in cell genesis. It is thus possible to affect the cell genesis from stem cells or progenitor cells and thus inducing the genesis of neurons and/or glial cells after either neuronal, oligodendroglial or glial cell loss in the CNS or peripheral nervous system (PNS), or to prevent the normal age related deterioration of said cells in the CNS or PNS.

In particular, the invention herein is based on the unexpected finding is that a specific number of pulses per day and other parameters of using rTMS also will increase neurogenesis in the hippocampal area in healthy humans that do not have any brain damages or diseases.

The term “rTMS” is in the present invention typically used to indicate a method for magnetic stimulation of biological tissue with pulsed magnetic fields.

In the invention, the typical range of stimuli frequency in the using rTMS in humans is 1-100 Hz with 100-3000 pulses given at each stimuli session and a stimulus intensity of 1-300 Ampere per microseconds. The optimum parameters to stimulate neurogenesis in the hippocampal formation of dentate gyrus are 20-100 Hz, and preferably more than 200 pulses, and most preferably, more than 1000 pulses given at each stimuli session and a stimulus intensity of 10-100 Ampere per microseconds.

The maximum frequency that may be used in the invention without the risk of causing side effects in healthy humans is 100 Hz unless special attention is given to reduce the intensity or number of pulses, especially in cases where the person to be given rTMS has a family history of epileptic disease.

Three to four weeks is the time considered needed for maturation and integration of progenitor cells into functionally working granule neurons.

The features of the present invention will be more clearly understood by reference to the following examples, which are not to be construed as limiting the invention.

### EXAMPLE 1

Adult healthy male Sprague-Dawley rats weighting 350-450 g are stimulated once daily with 200 or 1000 rTMS pulses for 14 consecutive days. A 20 Hz stimulus frequency is delivered from a stimulator (Dantec by Dantec Dynamics Ltd. Bristol, U.K.). The waveform is biphasic with a pulse width of 280 microseconds and a stimulus intensity of 98 Ampere per microseconds. A figure-eight coil (winding radius 5 cm) is placed on the awaken rat at the vertex of the skull and the coil is held in direct physical contact to the animal's head. The rats are held by hand and the handle of the figure-eight coil is placed parallel to the vertebral column of the rat. The animals are allowed to adapt to the experimental procedure during a ten day period before the actual experiments are started. During the adaptation period the animals are exposed to gradually longer times in the experimental situation to get used to the rTMS stimuli handling. It is found that after the period of adaptation the animals cooperate well with daily rTMS treatment without resistance and showing no signs of unspecific stress responses. The sham treated animals are subjected to identical handling as the rTMS treated animals except that the sham stimulations are performed with the coil held at 10 cm above the head. The sham treated rats receive 1000 sham pulses daily. The control animals are kept in their cages in the same animal housing room as the sham treated and rTMS treated groups during the whole experimental period. No differences in body weight gain are observed between the different groups during the course of experiments.

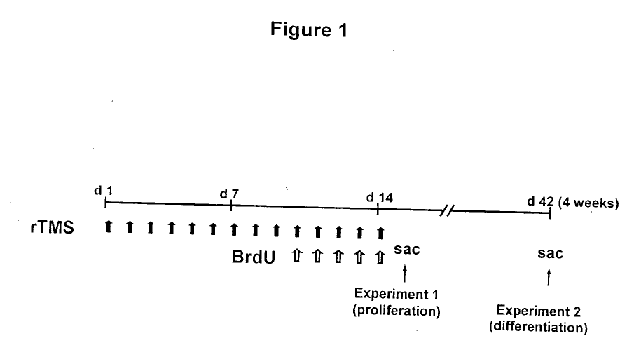

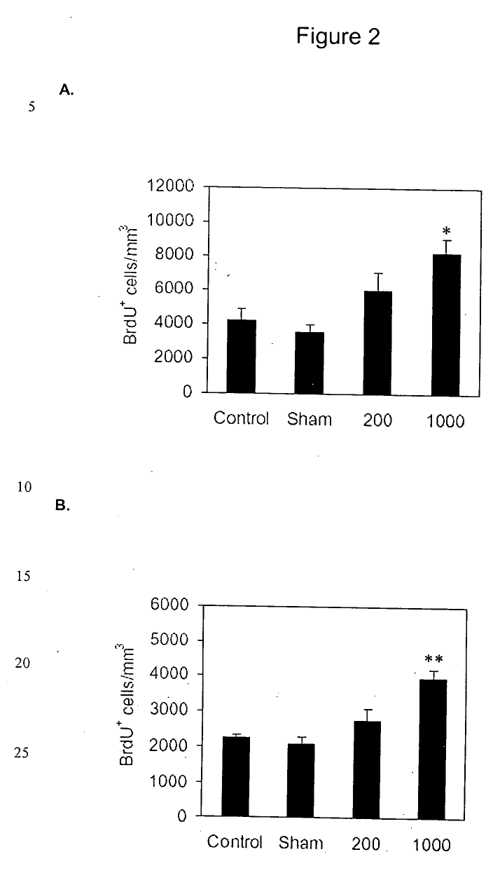

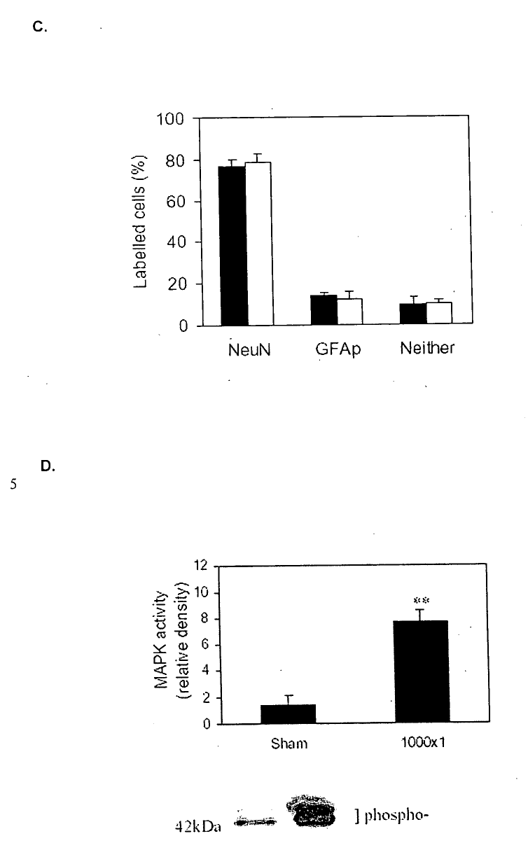

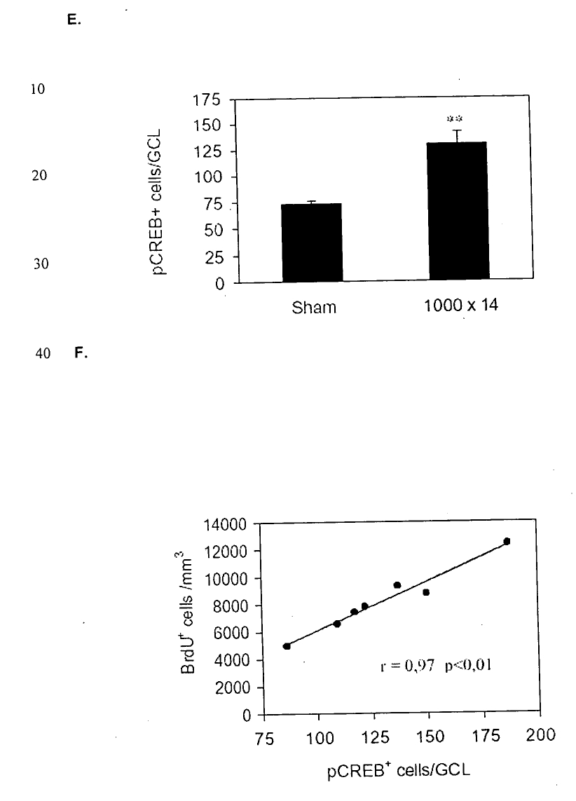

A magnetic stimulator giving a stimuli frequency of 20 Hz and a peak B-field of 1.6 T is used at the surface of a figure-eight coil to give rats a daily treatment with rTMS. In the first experiment, the proliferation of neuronal progenitors is analyzed in the dentate gyrus of the hippocampus after 14 days of daily rTMS treatments of the rat brain. The progenitor proliferation is known to occur in the subgranular zone (SGZ) of the dentate gyrus, which is the border between the granule cell layer (GCL) and the hilus. From the SGZ the progenitors migrate into the GCL where they mature into different neuronal cell types, primarily becoming neurons or astrocytes. The subgranuler progenitor proliferation is here analyzed using the thymidine analog bromodeoxyuridine (BrdU) as a marker for dividing cells. The rats are divided into four experimental groups including; two control groups consisting of one control for baseline neurogenesis in untreated animals and one sham stimulated group, one group receiving 200 rTMS stimuli pulses per day, and one group receiving 1000 rTMS stimuli pulses per day. The number of newly generated cells in the SGZ is determined by BrdU-injections given over 5 consecutive days in the end of the treatment period. Brains are taken for imunohistochemical analysis one day after the last rTMS treatment (for experimental paradigm see “Experiment 1” in FIG. 1). Most of the BrdU-immunoreactive cells in the SGZ are found in clusters, with irregularly shaped nuclei and rough patterns of BrdU-staining, which are characteristics of immature cells undergoing division. Analysis of the number of BrdU-labeled cells in this experiment demonstrates that 14 days of rTMS treatment has a significant effect within the groups (ANOVA F=7.32; p<0.001). Further comparisons (Scheffé test) show that 1000 rTMS stimuli per day significantly increases the number of BrdU-positive cells in the dentate gyrus relative to control (p<0.01) and sham treated animals (p<0.005). In animals subjected to 1000 daily rTMS pulses, the number of BrdU-immunoreactive cells in the SGZ increases to 8187±889 cells/mm3 in comparison to 4160±736 and 3579±398 cells/mm3 (n=7.7.7; mean±SEM) in the control and sham treated groups, respectively. A small but non-significant increase to 6004±1033 BrdU-positive cells/mm3 (p=0.231) is seen in the 200 rTMS stimuli group compared to sham treated animals (n=7; mean±SEM). There is no significant difference between the controls and sham treated animals (p=0.948).

Rat models have been used, for example in experimental mazes, since at least the early 20th century to better understand memory functions of humans. Thousands of studies have examined how rats run different types of mazes, from T-mazes to radial arm mazes to water mazes. These maze studies are used to study spatial learning and memory in rats. Maze studies helped uncover general principles about learning that can be applied to many species, including humans. Today, rat models are accepted methods predicting various conditions affecting learning and memory in humans.

### EXAMPLE 2

The data from Experiment 1 shows an increase in hippocampal progenitor proliferation from rTMS stimulation the highest stimuli dose as compared to control and sham treated animals. We furthermore wanted to know if this increase in proliferation of progenitors results in a higher number of newborn cells that persists with time, and whether this will give rise to an increase of mature neurons in the hippocampus at a later stage. Three to four weeks is the time considered needed for maturation and integration of progenitors into functionally working granule neurons. In Experiment 2, four groups of rats are subjected to the same rTMS treatments as described above and the animals are then allowed to live for four weeks after the last rTMS stimulation (see paradigm in FIG. 1). In the first part of this experiment the brains are analyzed to find the amount of BrdU-positive cells in the GCL of hippocampus. In these animals the BrdU-immunoreactive cells are found throughout the whole GCL, and the nuclei show a regular and smoothly rounded shape with an even BrdU-staining. One-way ANOVA reveals a strong significance within groups (F=13.19; p<0.0001) and further analysis (Scheffés test) shows significant increases in numbers of BrdU-positive cells in the 1000 stimuli group to 3936±246 cells/mm3, as compared with sham and control values of 2083±184 (p<0.0005) and 2238±96.5 (p<0.001) cells/mm3, respectively (n=5.5.5; mean±SEM). Similar to the outcome of the rTMS treatment in Experiment 1, no significant increase can be detected in BrdU-labeling within the 200 stimuli group after four weeks; 2730±331 cells/mm3 (n=5; mean±SEM p=0.307 vs. sham), and no difference is found between control and sham treated rats (p=0.973).

In a second part of Experiment 2 the number of BrdU-immunoreactive cells are studied in relation to the expression of cell type specific markers for neurons and astrocytes using confocal microscopy analysis of cells that were initially formed during the rTMS treatment period. The majority of the surviving BrdU+ cells in the GCL express the mature neuronal marker NeuN (76.7±3.3 and 78.1±4.6% for sham and rTMS 1000 treated animals, respectively as compared to co-localized immunoreactivities for BrdU and the astrocytic marker GFAp (14.1±1.1% and 12.3±3.7% for sham and rTMS 1000 treated animals, respectively (n=5.5; mean±SEM). Approximately 10% of the BrdU± cells in both groups show no co-localized staining with any of the markers. These relative numbers of phenotypic characteristics do not differ significantly between the sham and rTMS stimulated groups. Since the number of BrdU-positive cells are elevated four weeks after the rTMS treatment, together with the data demonstrating similar and high numbers of newly formed neurons in the sham and rTMS stimulated groups, it is concluded that the rTMS-induced progenitor proliferation leads to an increase in hippocampal neurogenesis.

### EXAMPLE 3

We next investigate if some intracellular signalling pathways known to be involved in neurogenesis in rats and believed by experts to be involved in humans could be stimulated by rTMS treatment. The p44 and p42 MAP kinases (Erk1 and Erk2) function in a protein cascade that plays a critical role in the regulation of cell growth. When rats are treated with 1000 pulses of rTMS there is an induction of p44/p42MAPK protein levels from 1.36±0.78 to 7.69±0.87 (relative density; n=5.5; mean±SEM; p<0.001, t-test). Phosphorylated cAMP response element binding protein (pCREB) is a transcription factor acting downstream of MAPK, and which is recently demonstrated to be involved in the regulation of hippocampal neurogenesis 13,14. In particular, the cAMP-CREB cascade has been shown to be involved in the up-regulation of neurogenesis from antidepressants including ECT (M. Nibuya, Nestler E J, Duman R S. J. Neurosci. 16(7):2365 (1996)). One of the target genes for pCREB is brain derived neurotrophic factor (BDNF). Dentate gyrus levels of BDNF have previously been shown to be increased by chronic rTMS treatment (M. B. Müller, Toschi N, Kresse A E, Post A, Keck M E. Neuropsychopharmacology (2):205 (2000)) and an increase in pCREB from rTMS stimulation has been observed in rat retinal cells (Ji R R, Schlaepfer T E, Aizenman C D, Epstein C M, Qiu D, Huang J C, Rupp F. Proc. Natl. Acad. Sci. U.S.A. 95(26):15635 (1998)) which made pCREB an interesting candidate for being regulated by rTMS in the hippocampus. A strong immunoreactivity for pCREB protein is found in the studies herein to be localized to cells in or adjacent to the SGZ. Treatment with rTMS 1000 stimuli for 14 days increases the number of pCREB immunoreactive cells from 73.1±2.9 in sham treated animals to 130±12.3 pCREB+ cells per dentate gyrus section (n=7.7; mean±SEM; p<0.005). Interestingly, there is a significant correlation between the pCREB immunoreactivity and the increase in BrdU incorporation for the seven animals in the 14 days rTMS 1000 experiment (correlation factor r=0.97; p<0.01).

### EXAMPLE 4

The aim of this study is to evaluate the efficacy of rTMS in memory improvement in healthy and voluntary humans under double-blind conditions compared with a sham-treated control group. We hypothesized that a specific dose of rTMS would within a certain number of weeks increase the neurogenesis in the hippocampal area of the test persons and show improvement in memory compared to the sham control based on our earlier studies in rats.

Methods

Forty healthy and voluntary persons are recruited. Patients are randomized to 4 treatment arms (n=10 each) via sealed envelopes opened immediately before commencement of the first session by the clinician administering the rTMS. The four experimental groups including one sham stimulated group are; one group receiving 100 rTMS stimuli pulses per day, the second group receiving 200 rTMS stimuli pulses per day, and the third active group receiving 1000 rTMS stimuli pulses per day. The rTMS stimulation is given once a day for 14 consecutive days. A 20 Hz stimulus frequency is delivered from a stimulator (MagPro x100, by Medtronic). The waveform is biphasic with a pulse width of 280 microseconds and a stimulus intensity of 100% of the motor threshold (MT). Motor threshold for the contra-lateral (right) abductorpollicisbrevis (thumb muscle) is determined by placing the coil over the optimal area of the left motor cortex and gradually increasing stimuli intensity to induce thumb movements. Then the figure-eight coil (winding radius 5 cm) is placed on the awakened person's head at a place on the skull representing left pre frontal cortical area and the coil is held in direct physical contact to the person's head. The sham group is given the same proceeding, including placing a sham rTMS coil on their skull, to simulate the sound of an active coil.

Test persons and raters are blind to treatment, but the clinician administering rTMS is aware of the treatment group. Patients are carefully and repeatedly instructed not to provide the raters with any information that would allow un-blinding of group. The primary outcome measure for the study is the memory effects. All test-persons are assessed at baseline and after four weeks after last rTMS administration via the Extended Rivermead Behavioural Memory Test (EERBMT) (de Wall C et. al., The Extended Rivermead Behavioural Memory Test, Memory. June 1994;2(2):149-66.) Because memory performance can only be tested by face-to-face, structured interviews and all other information is collected through face-to-face interviews. After obtaining consent from participants, the investigator collects demographic and mental status data to check for eligibility. If a participant is eligible for the study, the interview continues. The average length of an interview is 65 min.

Regarding illness and medication use, participants are asked to list any illness episodes that required visits to a physician during the last year and any chronic afflictions, such as hypertension or diabetes. Participants are asked to list any prescription and nonprescription drugs that they were currently taking. Persons suffering from any disease or taking any drugs for treatment of any disease are excluded from the trial.

The Extended Rivermead Behavioural Memory Test (RBMT) is an objective measurement of everyday memory, that is, the memory skills necessary for functioning in normal life. The RBMT consists of four parallel tests (A, B, C, and D), each with 12 test components. The 12 subtests include first name, last name, story (immediate and delayed), hidden belonging, appointment, route (immediate and delayed), message, faces, object pictures, and orientation. The reliability of the RBMT was established by parallel-form reliability (Wilson, Cockburn, Baddeley, & Hiorns, 1991). The correlations between Version A and Versions B, C, and D were 0.86, 0.83, and 0.88, respectively. Construct validity was evaluated as the correlation (0.75) between the RBMT scores and the number of memory lapses (Wilson et al., 1991). The RBMT was originally designed to detect memory impairment in patients with brain damage, therefore a ceiling effect may occur for normal adults. To adjust for this effect, de Wall et al. (1994) made the test more difficult by doubling the testing material. Versions A and B were combined, as were versions C and D, to form the Extended Rivermead Behavioural Memory Test (ERBMT), which is used in the current research.

Data Analysis

Descriptive statistics are used to describe the magnitude of each variable, and inferential statistics (correlation coefficient and multiple regression) is performed to examine the relationship between dependent and independent variables. For data analysis, gender is coded as male=1 and female=0.

Results

Table 1 presents the 12 subscale scores and total scores for the memory test.

TABLE 1
Extended Rivermead Behavioural Memory Test (N = 40)

| | | | After four weeks: | | |
|---|---|---|---|---|---|
| | At baseline: | | | | Theoretical |
| Variable | M (SD) | Range | M (SD) | Range | Range |
| Story immediate | 8.21 (3.17) | 2-18 | 9.32 (3.1) | 2-18 | 0-21 |
| Story delayed | 7.13 (3.02) | 0-16 | 7.92 (2.83) | 0-16 | 0-21 |
| Picture | 18.40 (2.03) | 8-20 | 18.38 (2.46) | 8-20 | 0-20 |
| Face | 3.70 (1.28) | 0-5 | 3.94 (1.16) | 1-5 | 0-5 |
| Route immediate | 4.85 (.42) | 3-5 | 4.87 (.4) | 3-5 | 0-5 |
| Route delayed | 4.90 (.33) | 3-5 | 4.90 (.39) | 3-5 | 0-5 |
| Message immediate | 4.35 (.78) | 2-5 | 4.78 (.57) | 2-5 | 0-5 |
| Message delayed | 4.84 (.49) | 3-5 | 4.89 (.41) | 3-5 | 0-5 |
| Orientation | 12.76 (.43) | 11-13 | 12.66 (.48) | 11-13 | 0-13 |
| Name | 2.70 (1.45) | 0-4 | 3.67 (1.03) | 0-4 | 0-4 |
| Appointment | 2.58 (1.52) | 0-4 | 3.27 (1.21) | 1-4 | 0-4 |
| Belongings | 6.04 (2.03) | 2-8 | 6.97 (1.74) | 2-8 | 0-8 |
| ERBMT | | | | | |
| Standardized total | 33.26 (5.17) | 21-44 | 38.49 (4.93) | 23-44 | 0-44 |

Table 2 presents the correlation coefficients of the predictor variables and the dependent variable. To test the individual relationships of the independent variables (sham, 100, 200, 1000 rTMS pulses per day), and demographic and control variables (age, gender) with changes of the dependent variable, Pearson's correlation coefficients are calculated. Among the four independent variables, only 200 and 1000 rTMS pulses per day is significantly correlated with memory function.

TABLE 2
Correlation Coefficients (N = 40)

| | Dependent Variable |
|---|---|
| Predictor Variable | ERBT |
| Sham administration | −0.18 |
| 100 rTMS pulses per day | −0.13 |
| 200 rTMS pulses per day | 0.11* |
| 1000 rTMS pulses per day | 0.45** |
| Age | −0.54*** |
| Gender | 0.07** |

*p < .05.
**p < .01.
***p < .001.

Discussion

The trial confirms that there is a need for a certain dose of rTMS, i.e. number of pulses to be more than 100 per day for an administration period to show positive effect on memory on healthy individuals when there has been sufficient time for maturation and integration of progenitors into functionally working granule neurons.

### EXAMPLE 5

Comparable studies on persons having symptoms of burnout indicate that the stress-mediated decrease in adult neurogenesis leading to a decreased ability to cope with stress through decreased hippocampal function can be counteracted by using similar doses and methods of applying rTMS as described in example 4.

What is claimed is:

1. A method of improving memory in a healthy adult person, comprising treating the brain of the healthy person with a session of repetitive transcranial magnetic stimulation pulses having a stimuli frequency, number of pulses, and intensity sufficient to induce an electric field in the brain of the healthy adult person to modulate a characteristic of the brain selected from the group consisting of proliferation, differentiation and migration of neuronal stem cells or progenitor cells in the central nervous system.

2. The method of claim 1, wherein the stimuli frequency is 1-100 HZ, with 100-3000 pulses at each session, and a stimulus intensity of 1-300 Ampere per microsecond.

3. The method of claim 2, wherein the stimuli frequency is 20-100 HZ, with 1000-3000 pulses at each session, and a stimulus intensity of 10-100 Ampere per microsecond.

4. The method of claim 1, wherein the adult is treated once per day for three to four weeks.

5. The method of claim 1, wherein the treatment is performed using a magnetic stimulator comprising a hand-held coil which is held near the head of the adult.

6. The method of claim 1, wherein the treatment results in increasing the number of neurons in the brain.

7. The method of claim 1, wherein the characteristic of the brain that is modulated comprises hippocampal formation of dentate gyrus.

8. A method of aiding in reduction of stress-related symptoms in a healthy adult person while improving the memory of the healthy adult person, comprising treating the brain of the healthy person with a session of repetitive transcranial magnetic stimulation pulses having a stimuli frequency, number of pulses, and intensity sufficient to induce an electric field in the brain to modulate a characteristic of the brain selected from the group consisting of proliferation, differentiation and migration of neural stem cells or progenitor cells in the central nervous system.

9. The method of claim 8, wherein the stress-related symptoms comprise burnout.

10. The method of claim 8, wherein the stimuli frequency is 1-100 HZ, with 100-3000 pulses at each session, and a stimulus intensity of 1-300 Ampere per microsecond.

11. The method of claim 10, wherein the stimuli frequency is 20-100 HZ, with 1000-3000 pulses at each session, and a stimulus intensity of 10-100 Ampere per microsecond.

12. The method of claim 8, wherein the adult is treated once per day for three to four weeks.

13. The method of claim 8, wherein the characteristic of the brain that is modulated comprises hippocampal formation of dentate gyrus.

14. A method of stimulating neurogenesis in the brain of a healthy human, comprising treating the human with repetitive transcranial magnetic stimulation.

15. The method of claim 14, wherein the treating comprises a session of repetitive transcranial magnetic stimulation pulses having a stimuli frequency, number of pulses, and intensity sufficient to induce an electric field in the brain of the healthy adult person to modulate a characteristic of the brain selected from the group consisting of proliferation, differentiation and migration of neuronal stem cells or progenitor cells in the central nervous system.

16. The method of claim 15, wherein the stimuli frequency is 1-100 HZ, with 100-3000 pulses at each session, and a stimulus intensity of 1-300 Ampere per microsecond.

17. The method of claim 16, wherein the stimuli frequency is 20-100 HZ, with 1000-3000 pulses at each session, and a stimulus intensity of 10-100 Ampere per microsecond.

18. The method of claim 14, wherein the adult is treated once per day for three to four weeks.

19. The method of claim 14, wherein the treatment results in increasing the number of neurons in the brain.

20. The method of claim 14, wherein the characteristic of the brain that is modulated comprises hippocampal formation of dentate gyrus.
